(19) United States
(12) Patent Application Publication
(10) Pub. No.: US 2022/0176141 A1
(43) Pub. Date: Jun. 9, 2022

(54) Device And Method For Wireless Microstimulation

(71) Applicant: The Regents of the University of Colorado, a body corporate, Denver, CO (US)

(72) Inventors: Anthony V. Caparso, North Ridgeville, OH (US); Richard J. Davis, Hillard, OH (US); Malik Kahook, Denver, CO (US); Naresh Mandava, Denver, CO (US)

(21) Appl. No.: 17/600,855

(22) PCT Filed: Apr. 1, 2020
(86) PCT No.: PCT/US2020/026260

§ 371 Date: Oct. 1, 2021

Related U.S. Application Data
(60) Provisional application No. 62/827,391, filed on Apr. 1, 2019.

(51) Int. Cl. A61N 2/00; A61N 2/02

(52) U.S. Cl. CPC A61N 2/006; A61N 2/02

(57) ABSTRACT

Systems and methods for wireless neural stimulation are presented. A microstimulator comprising a highly magnetic permeable material is implanted in the tissue of a living body. A wearable external controller creates a time-varying magnetic field that extends to the microstimulator in the tissue. The microstimulator re-shapes and boosts the time-varying magnetic field in the area surrounding the microstimulator, causing neural stimulation in the area around the microstimulator. A physician programmer device is also presented that allows a physician to program the wearable external controller.

### RELATED APPLICATIONS

This application claims the benefit of priority to U.S. Provisional Patent Application Ser. No. 62/827,391, filed on Apr. 1, 2019, which is hereby incorporated by reference in its entirety.

### TECHNICAL FIELD

Various embodiments disclosed herein generally relate to neural stimulation in a living body. More specifically, the embodiments of the present invention relate to wireless neural stimulation with a wearable controller and an implantable device.

### BACKGROUND

The human nervous system utilizes electrical signals to achieve many functions, including sensory input, muscle movements, memory, thoughts, action, involuntary and voluntary control of the visceral function, and controlling blood flow through the heart and circulatory systems. These electrical signals are represented as potentials (voltages) throughout the body and are created by ions, not electrons. These ion-channel mediated signals can be initiated and modulated by electrical fields that originate within or from outside the body. Using the theories of electromagnetism, electric fields can be generated using modulated magnetic fields, referred to a magnetic stimulation.

The signals inside the body are controlled via action potentials that are frequency modulated. An action potential is a rapid rise and subsequent fall in voltage or potential across a cellular membrane. These action potentials act as an information exchange from one cell or organ to the another and occur mostly along nerve fibers. The action potentials can occur repeatedly, creating a pulse train. The intensity of the reaction (e.g. nerve response, muscle contraction, etc.) can be related to the pulse rate of the action potentials. The end effect of the pulse train of action potentials is dependent on the origin and frequency of the pulse train. Action potentials are generated throughout the body and brain, in the central and peripheral nervous systems, and within/at each visceral organ, and muscle. Various devices and methods have been developed to mimic and/or augment these naturally occurring action potentials to achieve a variety of therapeutic responses for individual disease states. To accomplish these benefits, neuromodulation devices induce electric fields at the location where the action potential needs to be created, augmented or blocked for the treatment of the disease state. For example, external magnetic stimulators create a time-varying magnetic field from an external coil, which in turn generates an electric field within the body. When the electric field is induced on a portion of the nervous system, it causes a change in the transmembrane potentials, which can cause depolarization or hyperpolarization, which alters the pulse train generated from the nervous system and hence the effect of those signals on the end processes.

Numerous such neuromodulation devices have been developed. For example, implanted stimulation electrodes, which are implanted at the targeted location and are connected to electronics that control the electrical fields generated by the implanted neurostimulator. These types of systems can be externally powered or include an implanted battery (rechargeable or non-rechargeable). The electrodes can be close to the power source or can be connected through wires to the power source which may be implanted at a different location within the body. Implanted systems can be highly targeted but can also be highly invasive and unstable due to electrode movement and breakages due to body movement. In addition, with additional incision sites and subcutaneous tunneling, infections can be increased and cause for the systems to be explanted. Spinal cord stimulation, deep brain stimulation, hypoglossal nerve stimulation, and vagal nerve stimulation are examples of implanted stimulation electrode systems.

Magnetic stimulators produce changing magnetic fields external to the body through a coil positioned on the outside of the body to generate electric fields within the body that alter neuronal signals using Faraday's law. Magnetic stimulation systems have gained regulatory approvals for treatment of major depression, neuropathic pain, and headaches. These systems may include one or several coils to better target the therapy or provide better penetration into the body. Magnetic stimulation is non-invasive, but highly unpredictable and that can lead to low efficacy because the stimulation is not targeted or is limited by depth of penetration of the magnetic fields. Transcranial magnetic stimulation (TMS) is an example of a magnetic stimulation system.

Repetitive transcranial magnetic stimulation (rTMS) uses a magnet to activate the brain. rTMS has been studied for the treatment of depression, psychosis, anxiety, and other disorders. Unlike electroconvulsive therapy (ECT), in which electrical currents are used to stimulation a generalized portion of the brain, rTMS can be more targeted to a specific site or area in the brain. The effects of magnetic stimulation to the brain cause temporary disruption of neural signals. However, due to the challenges of precise placement of the coils, and body position of the patient, the treatments can vary in efficacy and is often unrepeatable within the same patient. Additionally, stimulation intensity is limited to a transient and narrow range between therapeutic effect and discomfort for the patient. The systems also require temperature control to prevent overheating. Magnetic stimulators can overheat prematurely due to the power required to generate the magnetic fields to produce the electric fields with the body. Therefore, magnetic stimulators are typically limited to a few seconds of stimulation followed by long periods of cooling. If the power was reduced to prevent overheating, the stimulation effects would be too weak to have a therapeutic effect.

In addition, magnetic stimulators can only induce electric fields that are strong enough to evoke action potentials within a few centimeters of the coil. This requires a significant amount of power, and current within the coil to generate the necessary electric fields within the body. The high voltage is required to change the current in the coil quickly, and the high currents in the coil are required to induce a sufficient electrical field in the body that achieves the desired effects. With these current issues with external magnetic stimulators, improvements are needed for the potential therapies to become viable, predictable, efficacious and cost effective.

Transcutaneous stimulators use electrodes placed on the skin surface to cause electrical current to flow into the body from one electrode to the other. Many TENS systems are available over the counter for muscle stimulation, pain, and other therapeutic modalities. Transcutaneous stimulation systems are also non-invasive but challenging to target and control due the unpredictable nature of the multiple current paths through the skin with varying intensities. Electro convulsive therapy (ECT) and transcutaneous electrical neural stimulation (TENS) are examples of transcutaneous skin electrode systems.

### OVERVIEW

Systems, methods and apparatuses for providing neuromodulation or neurostimulation to various nerves in a living body are disclosed herein. In one example, a system for stimulating nerves in a living body is disclosed. The system includes an implantable microstimulator which is configured to be positioned at least partially within the tissue of the living body, the microstimulator including a magnetic focal element which is electrically isolated from the tissue. The system further includes a portable external controller which is configured to be worn on and external to the living body, the portable external controller including a processor, a power source, a driver circuit and an inductive coil. The processor is configured to control the power source and inductive coil to produce a time-varying magnetic field extending to the implantable microstimulator positioned within the tissue. The magnetic focal element is configured to increase the time-varying magnetic field in a space proximate to the magnetic focal element. The time-varying magnetic field causes an elevated electrical current density in a stimulation area of the tissue proximate the implantable microstimulator, causing neural stimulation within the targeted stimulation area of the tissue.

In another example, a method for stimulating nerves in a living body is disclosed. The method includes providing an implantable microstimulator which is configured to be positioned at least partially within the tissue of the living body, the microstimulator including a magnetic focal element which is electrically isolated from the tissue. The method further includes providing a portable external controller which is configured to be worn on and external to the living body, the portable external controller including a processor, a power source, a driver circuit and an inductive coil. The processor is utilized to control the power source and inductive coil to produce a time-varying magnetic field extending to the implantable microstimulator positioned within the tissue. The magnetic focal element increases the time-varying magnetic field in a space proximate to the magnetic focal element. The time-varying magnetic field causes an elevated electrical current density in a stimulation area of the tissue proximate the implantable microstimulator, causing neural stimulation within the stimulation area of the tissue.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the scope of the present invention. Accordingly, the drawings and detailed description are to be regarded as illustrative in nature and not restrictive.

### BRIEF DESCRIPTION OF THE DRAWINGS

Embodiments of the present technology will be described and explained through the use of the accompanying drawings.

FIG. 1 illustrates an electromagnetic system.

FIG. 2 illustrates a schematic representation of an electromagnetic system.

FIG. 3a illustrates an inductive element.

FIG. 3b illustrates an inductive element.

FIG. 4a illustrates a magnetic field.

FIG. 4b illustrates a magnetic field.

FIG. 5a illustrates an implantable element.

FIG. 5b illustrates an implantable element.

FIG. 6 illustrates a portion of the neural system of a human body.

FIG. 7 illustrates an external controller according to an embodiment of the disclosure.

FIG. 8 illustrates a system according to an embodiment of the disclosure.

The drawings have not necessarily been drawn to scale. Similarly, some components and/or operations may be separated into different blocks or combined into a single block for the purposes of discussion of some of the embodiments of the present technology. Moreover, while the technology is amenable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

The present disclosure relates to a system and methods for providing neuromodulation or neurostimulation to nerves or tissues in a living body. According to embodiments, this neurostimulation or neuromodulation is supplied to the sphenopalatine ganglia (SPG), the sphenopalatine nerve (SPN), the vidian nerve (VN), the greater and/or deep petrosal nerves or other nerves and or branches of the sphenopalatine ganglion for the treatment of disorders and diseases.

According to an embodiment, a neuromodulation system is intended to produce bilateral or unilateral stimulation of the SPG. The neuromodulation system may have the following components, but not limited to these components; a microstimulator, a patient handheld activation unit (controller) to activate and control the microstimulator, and a physician programming unit that communicates with the handheld activation unit (controller) and is configured to adjust the stimulation parameters associated with the microstimulator and to assess the microstimulator's functionality. The system may also include a mobile application that can interact with the patient activation unit (controller) and the physician programming unit.

The microstimulator described in this application includes the necessary components and materials to reshape and boost an external magnetic field and to harness that reshaped and boosted field to generate sufficient current density to stimulate the surrounding tissue. In this configuration, the microstimulator can include a material that has a high magnetic permeability within the microstimulator. Materials with high magnetic permeability include Mu-metals (nickel-iron soft ferromagnetic alloys, such as those shown on p. 354 of Introduction to Magnetism and Magnetic Materials), permalloy (nickel-iron magnetic alloy, shown on p. 337 of Introduction to Magnetism and Magnetic Materials, Second Edition by David C. Jiles, metglas (a thin amorphous metal alloy ribbon produced by using rapid solidification process, shown on p. 98 of Introduction to Magnetism and Magnetic Materials), other ferritic metals. The material properties can be selected based on the magnetic field generated and the amount of current density required for stimulation. The shape, position, and alignment of the material with in the microstimulator is also important as it relates to the specific magnetic field generated from the external device.

In an embodiment, the external device and microstimulator are positioned and configured to produce stimulation or disruption of neural signals passing through the autonomic nervous system, including the SPG, the SPN, or VN. PG or surrounding nerves or branches. The abnormal regulation of neural pathways, which may be a feature of the conditions described herein, can cause excitation or a loss of inhibition of those pathways, resulting in therapeutic benefit for the patient. In this embodiment, one or more microstimulators that consist of a highly magnetically permeable material are implanted directly on or adjacent to the SPG, SPN, and/or the VN of a patient. For purposes of clarity, in the following discussion it shall be assumed that a single microstimulator is implanted with the patient. However, it should be understood that multiple microstimulator may be implanted according to the invention.

The system will also include the use of an external device that is configured to produce a varying magnetic field. The magnetic field is generated using an efficient driver circuit that enables high voltage in the coils with low voltage battery power. The external device may be configured as a stand-alone product, such as a remote control, a key fob, as an attachment to a standard smart phone, as a wearable product, and/or a clip on to clothing such as a belt, hat, etc. In one embodiment, the external device may also have a digital interface to allow patient control of the stimulation, allow exchange of data about the therapy and provide alerts to the patient (timed or pushed alerts).

The external device may have one or multiple coil configuration to apply the appropriate magnetic field to the microstimulator. Each coil can be configured to drive multiple frequencies of the magnetic field and be electrically controlled to provided time varying and alternating magnetic fields that produce different types of electrical fields with the body, which will interact differently with the microstimulator.

In one embodiment, along with the microstimulator and external device, the system includes an external programming device. The programming device allows the physician to appropriately set up the external device to apply the right magnetic field to produce the right electrical field and appropriate interaction with the microstimulator to apply therapy to the patient.

The phrases “in some embodiments,” “according to some embodiments,” “in the embodiments shown,” “in other embodiments,” and the like generally mean the particular feature, structure, or characteristic following the phrase is included in at least one implementation of the present technology, and may be included in more than one implementation. In addition, such phrases do not necessarily refer to the same embodiments or different embodiments.

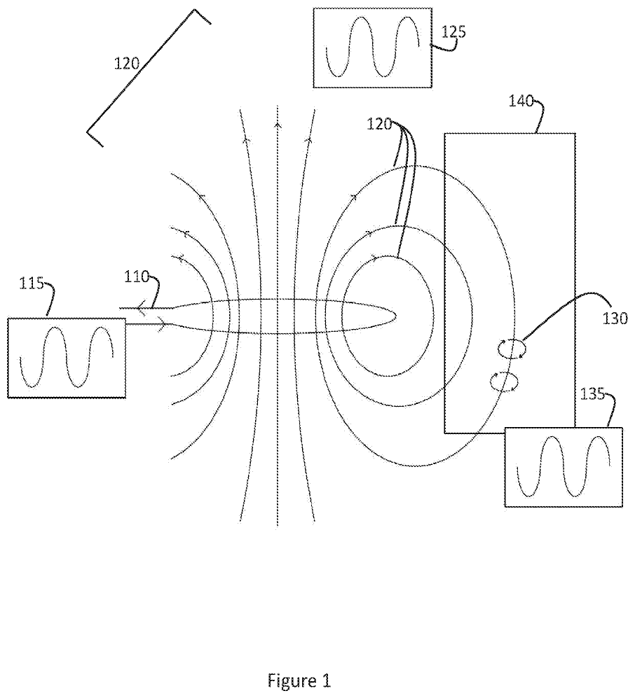

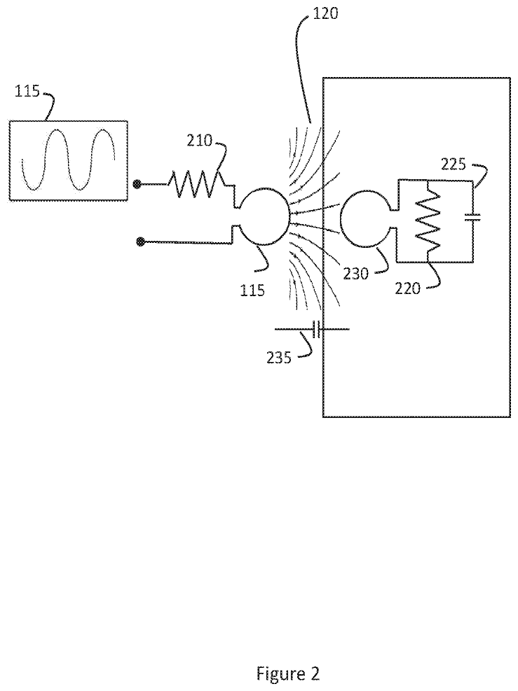

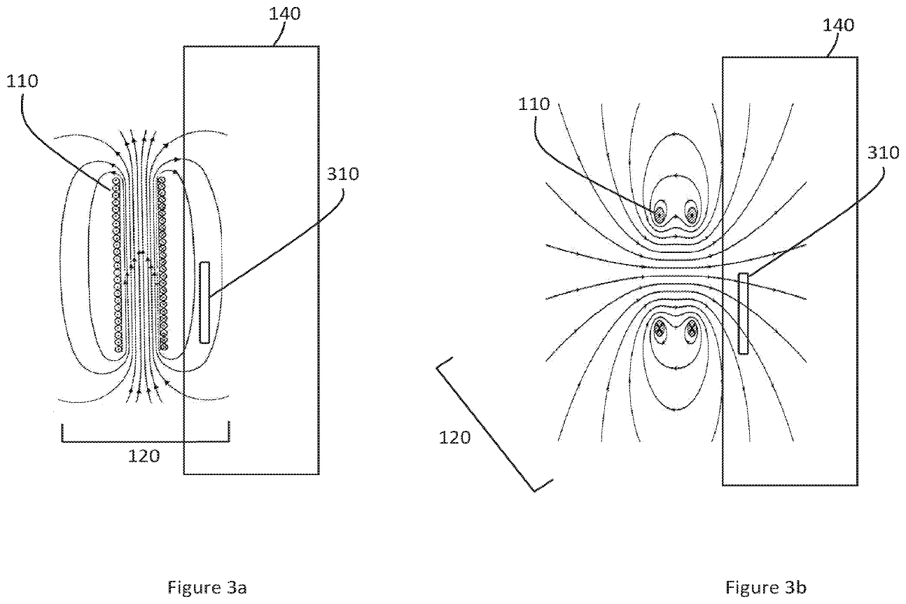

Now referring to the figures, FIG. 1 illustrates an electromagnetic system. The system includes elements external to, and elements internal to a living body 140. External to the living body 140 is a stimulation coil 110. The stimulation coil 110 can take many different forms, according to various embodiments. For example, the stimulation coil 110 can be conductive wire wrapped abound a ferromagnetic core. Alternatively, the wire may be wrapped around an inert core, or no core. Additionally, the wire may be wrapped in different ways around the core, particularly in relation to the living body 140, as will be discussed in more detail with reference to FIGS. 3a and 3b below. A time-varying electrical drive signal 115 is provided into the stimulation coil 110. The electrical drive signal 115 can be provided by a voltage or current source, for example. The electrical drive signal 115 can be a periodic wave signal, such as a sinusoidal signal, square wave or triangle wave, or a signal that varies over time with limited or no periodicity. As the electrical drive signal 115 is provided to the stimulation coil 110, a magnetic field 120 is created. This magnetic field 120 also has a magnetic field signal 125. The magnetic field 120 created by the stimulation coil 110 may be time-varying as well. For example, the magnetic field signal 125 may be periodic, or some other time-varying signal with limited or no periodicity. It should be understood that the magnetic field signal 125 can be determined and controlled by controlling the electrical drive signal 115 in conjunction with the stimulation coil 120. The magnetic field 120 permeates into the living body 140. The magnetic field signal 125 can create eddy currents within the living body 140, as will be discussed more in relation to FIG. 2. These eddy currents 130 can also have a time-varying eddy current signal 135. This eddy current signal 135 can also be a periodic signal, or a signal with limited or no periodicity. Again, it should be understood that this eddy current signal 135 can be determined by controlling the electrical drive signal 115 with knowledge of the inductive coil 110 and the living body 140.

Turning to FIG. 2, a schematic representation of the electromagnetic system is shown. Outside of the living body 140, the stimulation coil 110 is shown. The stimulation coil has a measurable inductance 215. This stimulation coil 110 is further electrically connected to a circuit having a resistance 210. The electrical drive signal 115 is applied to the circuit. This electrical drive signal 115 can be in various forms (i.e. a voltage source, a current source, etc.). The magnetic field 120 is shown permeating the living body 140. As discussed above, this magnetic field 120 also has a time-varying signal 125 (not shown). The capacitance of the link 235, eddy current 230, impedance of the body 220 and capacitance of the body 225 are not discrete electrical components, but rather characteristics of the living body 140 in a location of interest. The capacitance of the link 235, eddy current 230, impedance of the body 220 and capacitance of the body 225 can vary over time as well. For instance, as the level of hydration or salinity changes, these elements may vary.

According to an embodiment of the neuromodulation system comprises an external, handheld or wearable device containing a magnetic field generator and an implanted microstimulator that reshapes and focuses the electrical field generated with the body from the external magnetic field to stimulate a target nerve fiber, neuron, or ganglion. The neuromodulation system comprises a handheld or wearable device, which contains a stimulation coil 110 to produce the magnetic field 120, that is driven by a driver circuit and powered by a battery or other power source. The driver circuit may contain a processor to generate the electrical drive signal 115 to the stimulation coil 110 and to receive input to allow adjustments in parameters for the stimulation coil 110 by the user or physician, via a programming unit or via a smart device (phone, tablet, watch, etc.). The interaction between the external device that produces the magnetic field 120 and the programming system, patient or physician, can be controlled via Bluetooth, WIFI, or other similar wireless protocols. The external system can be handheld or attached to the body as a wearable device (e.g., watch, glasses, hat, belt, etc.) or maybe attached to clothing or other attire.

The current generated within the stimulation coil 110 (located within the external device) produces a changing magnetic field 120 that easily penetrates into the living body 140, including hard and soft tissue structures. By Faraday's law of electromagnetics, this changing magnetic field 120, induces an electric field with the body. In some embodiments, the induced electric field is configured for a larger area within the body or can be configured for a targeted, localized area to alter the neurological signals in the immediate location. The field interacts with the microstimulator to produce a reshaped and highly focused electrical charge around the microstimulator, which in turn will cause electrical stimulation of nearby neural structures.

The external device is configured to generate a magnetic field 120 that can interact with the inserted microstimulator. The inserted microstimulator must be placed within a conductive medium that can induce eddy current 130. The human body is a high conductive medium and capable of producing eddy currents generated from an external magnetic field. In this configuration, the external device can include an inductive coil. The magnitude of the magnetic field is proportional to the inductor coils amperage and number of wire turns within the coil as well as the control signal to create the magnetic field, such as the frequency and amplitude. The resulting magnetic field 120 generated from the inductive coil permeates the human body and when the field interacts with the magnetic permeable material, the material will reshape and boost the magnetic field to induce sufficient current density to cause stimulation of the tissue surrounding the microstimulator. The external device can use any means of creating the magnetic field.

Turning now to FIG. 3a, the stimulating coil 110 can be an axial inductive coil, in which the wires that make up the stimulating coil 110 are orientated 90 degrees to the direction of the magnetic permeable material within the microstimulator 310. The microstimulator 310 is shown inserted into the living body 140, and generally parallel to the surface of the living body 140. The orientation of the stimulation coil 110 to the surface of the living body 140 can vary from that shown here. The magnetic field 120 projects from the stimulating coil 110 into the living body 140 and to the microstimulator 310. The microstimulator 310 will affect the magnetic field 120, depending on the materials in the microstimulator 310 among other factors. This effect is not shown in FIG. 3a.

In FIG. 3b, the stimulating coil is a pancake inductive coil, in which the wires that make up the stimulating coil 110 are orientated parallel to the material within the microstimulator. The microstimulator 310 is again shown inserted into the living body 140, and generally parallel to the surface of the living body 140. The orientation of the stimulation coil 110 to the surface of the living body 140 can vary from that shown here. The magnetic field 120 projects from the stimulating coil 110 into the living body 140 and to the microstimulator 310. The microstimulator 310 will affect the magnetic field 120, depending on the materials in the microstimulator 310 among other factors. This effect is not shown in FIG. 3b.

In an embodiment, the external device may be configured with a variety of stimulation coils 110 connected to a variety of driver circuits. The coils may be configured with a number of turns, including 10-500 turns or about 40 to 200 turns or more. In general, with increased numbers of turns, the inductance 215 of the coil increases, which increases the voltage of the driver circuit but decreases the current needed to provide the magnetic field.

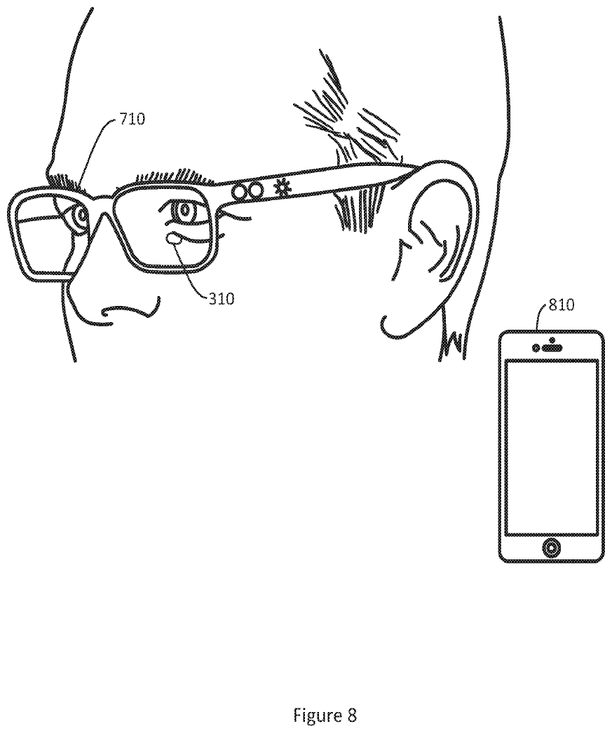

In an embodiment, the diameters of the coils are determined by the penetration depth needed for the induced electrical field. Some nerves are superficial to the skin surface, and other nerves are located more deeply with the body. The SPG is approximately 2-4 cm deep to the skin of the lateral cheek, and 2-3 cm deep to the anterior cheek, just medial to the lateral nose. In general, for good efficiency the diameter of the external coil may be approximately four times the penetration depth required. However, this can be optimized and configured for the application and using coil production techniques that increase surface area of the coil and allow for higher currents to be generated without causing additional heat from the current. These methods can include flex circuit coil designs using multiple layers to the flex design. In addition, in some embodiment, the stimulation coils can be configured with unique geometries including FIG. 8 coils, which have been shown to produce a stronger and deeper magnetic field depth. In an embodiment, the coil diameter is between 2 cm and 40 cm, and more specifically, between 2 cm and 10 cm.

In an embodiment, the stimulation coil 110 is configured to generate a magnetic field strength between 0.001 and 0.1 Tesla or more as needed in to induce a sufficient electrical charge at the microstimulator to stimulate nearby neuronal structures. The magnetic field strength may be smaller for narrower pulse widths because the voltage is proportional to the magnetic field time derivative. To generate these magnetic fields, the driver circuit needs to produce currents in the stimulation coils to drive 1-20 amperes or more specifically 0.2-5 amperes in short bursts for each stimulation pulse to the neuronal structure.

In some embodiments, the pulse width, frequency, burst rate, and amplitudes are defined by the stimulation parameters and are determined by the driver circuits. In one embodiment, the pulse width is between 10 and 1000 microseconds, and up to 2000 microseconds, frequencies between 1 and 10,000 Hz and amplitudes between 0.1 to 10 microamps, up to possible 20 microamp.

In an embodiment, the microstimulator 310 is configured with a highly magnetically permeable material. The material can have a magnetic permeability of between 150 and 1,000,000μ. According to an embodiment, the material may have a magnetic permeability greater than 500μ, or in some cases greater than 1,000μ, or over 10,000μ. The microstimulator material make up is configured to produce the appropriate amount of permeability to the externally applied magnetic field to cause an interaction between the material and the electrical field within the body. The interaction causes the electrical field to be increased in the immediate area of the microstimulator and causes the electrical field to be more focused within the body, allowing for electrical stimulation to occur immediately surrounding the microstimulator 310. The material interacts with the induced magnetic field 120 generated by an external controller and causes the magnetic field 120 to reshape, increase in intensity and cause significant amount of current density around the microstimulator 310. In general, a normal magnetic field 120 may not cause tissue stimulation in and of itself due to the properties of the magnetic field 120. However, when the material is added to the microstimulator 310, the same magnetic field 120 that was not capable of creating tissue stimulation can now cause stimulation due to the boosted and reshaped magnetic field caused by the magnetically permeable material within the inserted microstimulator 310.

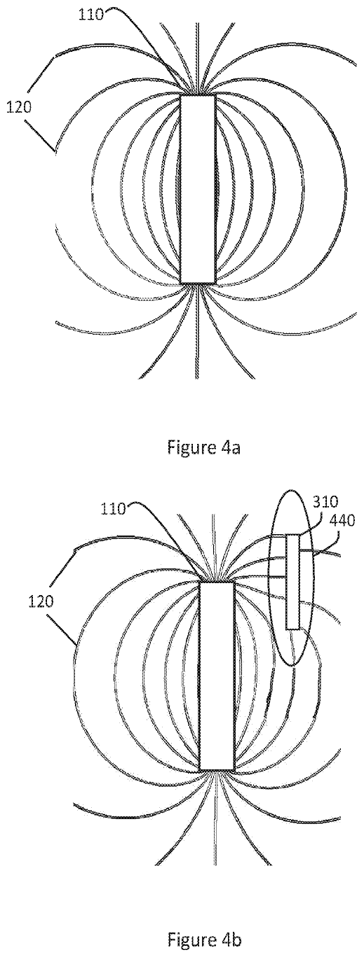

In FIG. 4a, a stimulation coil 110 is shown producing a magnetic field 120. In FIG. 4b, the same stimulation coil 110 is shown producing a magnetic field 120. In FIG. 4b, a microstimulator 310 containing a piece of highly magnetic permeable material has been added within the range of the magnetic field 120. Because of the microstimulator 310, the magnetic field 120 is modified to boost and reshape the magnetic field 120 in the area around the microstimulator 310.

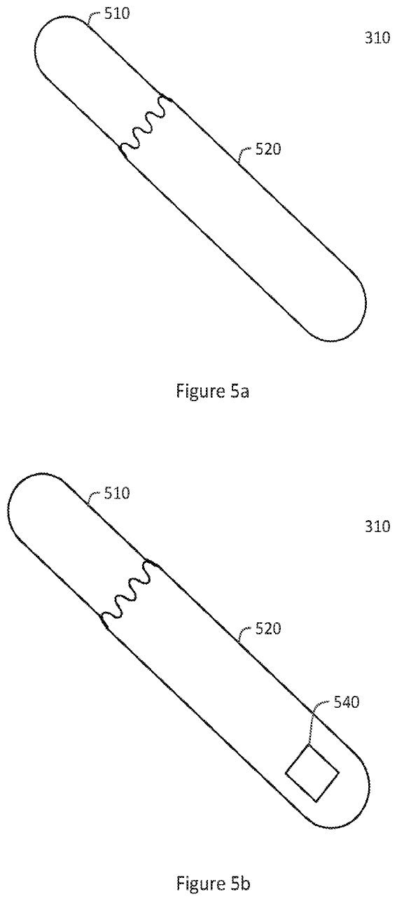

FIG. 5a shows the microstimulator 310 according to an embodiment. The microstimulator 310 may include an encapsulating material 520 around the highly magnetically permeable material 510. The encapsulating material 520 can be made of polyurethane, or other materials that have known biocompatibility with the body, and also have properties that do not allow metals or other chemicals to leach into the body from the microstimulator.

The shape of the microstimulator can vary. In an embodiment, the shape is configured to apply electrical stimulation to the SPG within the pterygopalatine fossa (PPF). The SPG varies in shape and position person to person, but in general the SPG is 1 to 2 mm in width, 2-6 mm in length and 1-3 mm thick. The SPG is typically located in the upper third of the PPF and in the posterior half of the PPF. In one embodiment, the microstimulator is sized to provide electrical stimulation to the SPG, configured to have a length of 3-8 mm, a diameter of 0.5 to 2 mm. Alternatively, in an embodiment, the microstimulator can be shaped as a 3 dimensional oval, with a thicker middle section, and thinner edges. In another embodiment, the microstimulator can be shaped like a cylinder that is bent into C configuration. In another embodiment, the microstimulator is configured as other shapes, including cylinders, 3D ovals, C configuration, hollow cylinders, or any combination of these or other configurations not described.

In some embodiments, the microstimulator is otherwise not anchored by any means within the anatomy. Because the microstimulator is placed within the PPF, a portion of the midfacial anatomy that does not move relative to other cranial or facial structures, the microstimulator is not subject to external forces that may cause dislodgement or movement of the microstimulator, other than severe trauma. In other embodiments, the microstimulator may include anchoring mechanisms, including small barbs or tines for example. The microstimulator may also include small micro-perforations that allow ingrowth of encapsulation tissue to anchor the device with the body of the patient.

As shown in FIG. 5b. in an embodiment, the microstimulator is configured to contain one or more electrically conductive elements, commonly referred to as electrodes 540. Each electrode 540 can be made of an electrically conductive material. In one embodiment, one or more electrodes 540 are can be made of platinum, platinum/iridium, palladium or another inert metal that is electrically conductive. The electrodes can be positioned between the distal portion of the microstimulator 310 and the proximal portion of the microstimulator 310, or throughout the geometry of the microstimulator. The electrodes, in this microstimulator are not used to conduct electrical current, instead they are used to reduce the induced magnetic field in areas in which stimulation of the tissue is not wanted. In one configuration, electrically conductive electrodes 540 can be placed on either end of the microstimulator, effectively allowing the magnetic permeable material 510 in the center of the microstimulator to produce significant current density and causing reduction in the magnetic field near the electrically conductive electrodes 540. The pattern of the magnetic permeable material 510 as well as the pattern of the electrically conductive material 540 can be varied to create the effect needed to cause localized stimulation of the target tissue. It should be appreciated that the exposed electrodes 540 can be positioned using any orientation around or within the microstimulator body from the distal portion to the proximal portion of the microstimulator body.

In an embodiment, microstimulator 310 may elute a NSAID, corticosteroids or other drugs, which will help to reduce inflammation of the surrounding tissue from the insertion of the device and help to promote healing and stable tissue interface after the insertion of the microstimulator. In other embodiment, the drug is eluted through the use of electrical energy, using iontophoresis methods. In this embodiment, small micro-currents are used to release and drive the compound or drug in precise areas and/or in precise amounts. In this embodiment, drug delivery can be done based on a pre-determined schedule or using biofeedback in a close loop manner.

The microstimulator 310 can be configured with multiple types of highly permeable magnetic materials. These materials can include, iron, silicon iron, permallowy, hipernik, supermalloy, mumetal, permendur, hipereo, metglas, ferrite, nickel, or supermendur for example. It should be noted, that other permeable materials may be used as well, as this list is not an extensive list. The materials can be layered or configured to produce different permeabilities throughout the microstimulator to change the way the microstimulator interacts, shapes, focuses and boost the resulting electrical fields within the body from the externally generated magnetic field generated from the external device. The materials can be aligned such that their natural dipoles are configured to be aligned or misaligned to provided increased or decreased permeability of the resulting material. One or more composite materials can be included into the same microstimulator, each configured to interact, reshape, focus and boost the electrical field differently or only interact with certainly frequency of electrical fields generated from the externally generated magnetic field.

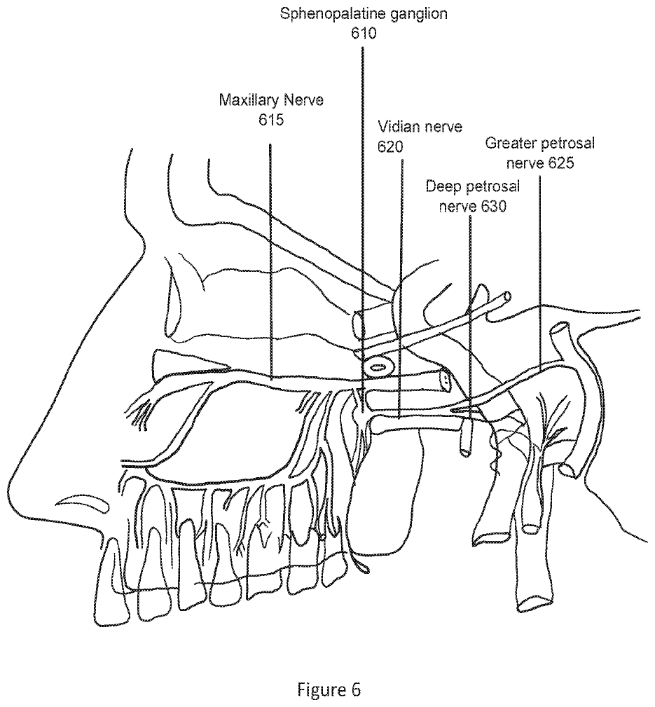

Turning to FIG. 6, A brief discussion of the pertinent neurophysiology is provided. The nervous system is divided into the somatic nervous system and the autonomic nervous system (ANS). In general, the Somatic nervous system controls organs under voluntary control (e.g., skeletal muscles) and the ANS controls individual organ function and homeostasis. For the most part, the ANS is not subject to voluntary control. The ANS is also commonly referred to as the visceral or automatic system. The ANS can be viewed as a “real-time” regulator of physiological functions that extracts features from the environment and, based on that information, allocates an organisms internal resources to perform physiological functions for the benefit of the organism, e.g., responds to environment conditions in a manner that is advantageous to the organism.

The ANS conveys sensory impulses to and from the central nervous system to various structures of the body such as organs and blood vessels, in addition to conveying sensory impulses through reflex arcs. For example, the ANS controls constriction and dilatation of blood vessels; heart rate; the force of contraction of the heart; contraction and relaxation of smooth muscle in various organs such as the lungs, stomach, colon, and bladder, visual accommodation; and secretions from exocrine and endocrine glands, etc.

The parasympathetic nervous system (PNS) is part of the ANS and controls a variety of autonomic functions including, but not limited to, involuntary muscular movement and glandular secretions from the eyes, salivary glands, bladder, rectum and genital organs.

The sphenopalatine ganglion (SPG 610), also called the pterygopalatine ganglion, is located within the pterygopalatine fossa (PPF). The PPF is bounded anteriorly by the maxilla, posteriorly by the medial plate of the pterygoid process and greater wing of the sphenoid process, medially by the palatine bone, and superiorly by the body of the sphenoid process. Its lateral border is the pterygomaxillary fissure, which opens to the infratemporal fossa.

The SPG 610 is a large, extra-cranial parasympathetic ganglion. The SPG 610 is a complex neural ganglion with multiple connections, including autonomic, sensory and motor. The maxillary branch of the trigeminal nerve and the nerve of the pterygoid canal, also known as the Vidian nerve (VN 620) sends neural projections to the SPG 610. The fine branches from the maxillary nerve, known as the pterygopalatine nerves or sphenopalatine nerves (SPN), form the sensory component of the SPG 610. The SPN pass through the SPG 610 and do not synapse. The greater petrosal nerve (GPN 625) (discussed below) carries the preganglionic parasympathetic axons from the superior salivary nucleus to the SPG 610. These fibers synapse onto the postganglionic neurons within the SPG. The deep petrosal nerve (DPN 630) (discussed below) connects the superior cervical sympathetic ganglion to the SPG 610 and carries postganglionic sympathetic axons that again pass through the SPG 610 without any synapses. The DPN 630 and the GPN 625 carry sympathetic and parasympathetic fibers, respectively. The greater and lesser palatine nerves are branches of the SPG that carry both general sensory and parasympathetic fibers.

The DPN 630 and the GPN 625 join together just before entering the pterygoid canal to form the VN 620. The DPN 630 is given off from the carotid plexus and runs through the carotid canal lateral to the internal carotid artery. It contains postganglionic sympathetic fibers with cell bodies located in the superior cervical ganglion. It then enters the cartilaginous substance, which fills the foramen lacerum, and joins with the greater superficial petrosal nerve to form the VN 620. The GPN 625 then passes through the SPG 610 without synapsing and joins the postganglionic parasympathetic fibers in supplying the lacrimal gland, the nasal mucosa, and the oral mucosa. The GPN 625 is given off from the geniculate ganglion of the facial nerve. It passes through the hiatus of the facial canal, enters the cranial cavity, and runs forward beneath the dura mater in a groove on the anterior surface of the petrous portion of the temporal bone. The GPN 625 enters the cartilaginous substance, which fills the foramen lacerum, and then joins with the DPN 630 to form the VN 620. The lesser petrosal nerve carries parasympathetic (secretory) fibers from both the tympanic plexus and the nervus intermedius to the parotid gland.

The VN 620 projects to the PPF through the vidian canal. The VN contains two of the three neural roots of the SPG 610, parasympathetic and sympathetic. The third neural root of the SPG 610 includes sensory fibers that derive from the second division of the trigeminal nerve, also called maxillary nerve 615. The maxillary nerve 615 connects to the SPG 610 through the SPN and this causes the SPG 610 to suspend form the maxillary nerve 615 within the PPF.

The VN 620 is housed within the Vidian canal, which is posterior to the SPG 610. The VN 620 connects to the SPG 610 and contains parasympathetic fibers, which synapse in the SPG 610, sensory fibers that provide sensation to part of the nasal septum, and also sympathetic fibers. The SPN are sensory nerves that connect the SPG 610 to the maxillary nerve 615. The SPN traverse through the SPG 610 without synapsing and proceed to provide sensation to the palate. The SPN suspend the SPG 610 in the PPF.

In some embodiments, the microstimulator is configured to be injected into the body via a very thin needles or catheter type device. The location of the SPG 610 with the PPF, allows for the device to be injected through the nasal cavity, through the lateral cheek using an infrazygomatic approach, transoral through a gingival buccal insertion, or through the greater palatine canal in the hard palate. The trans-nasal approach can be accomplished using an endoscopic procedure to enter the PPF through the sphenopalatine foramen and visualizing the structures with the PPG, including the foramen rotundum and the vidian canal/vidian nerve and placing the microstimulator along the VN 620 and next to the SPG 610. Using a working channel on the endoscope, the microstimulator can be placed through the endoscope directly or with a separate delivery tool or needle. In one embodiment, the microstimulator can be placed using a needle position above or below the zygomatic arch and utilizing fluoroscopy, place the needle within the PPG to deliver the microstimulator. In another embodiment, the microstimulator can be placed using traditional approaches to the PPG for blocking V2 (the maxillary division of the trigeminal nerve). Using a curved needle, pull the cheek back and locate the maxillary buttress and soft tissue behind the maxillary buttress, position the needle superiorly and medially toward the medial canthus of the ipsilateral eye. This procedure can be done under fluoroscopy as well. In another embodiment the microstimulator can be placed via the greater palatine canal. The greater palatine canal is located just medially to the second/third molar on the upper hard palate of the patient. The canal is an anatomical opening that leads directly to the PPF. Using a needle and also fluoroscopy, the microstimulator can be delivered directly within the PPF and next to the SPG 610. The microstimulator can be configured and shaped to fit with a needle or other delivery systems using any of these potential procedures. Many of these procedures only require topical or local anesthesia and therefore can be performed as an outpatient procedure, or within a typical surgical center, such as an advanced surgical center (ASC) or in the case of the endoscopic procedure, may be performed in an office setting.

According to an embodiment, the microstimulator is configured to be inserted into the PPF of a subject. The microstimulator consists of an elongated cylinder with an embedded highly magnetically permeable material or composition material made of several magnetically permeable materials. In this embodiment, the microstimulator also includes a material that has high magnetic permeability configured into a circular cross-section and position throughout the microstimulator length. The magnetic permeable material is configured to be completely insulated within a silicone material or polyurethane material, or other suitable material for long term tissue contact. In addition to the magnetic permeable material, the microstimulator includes two ring electrodes made of electrically conductive material and positioned more proximally and more distal to the magnetic material. The insulated and electrically conductive material is in contact with the tissue surrounding the SPG 610. The magnetic permeable material is also configured to interact, harness and boost the magnetic field within the PPF, causing significant increases in current density surround the microstimulator near the magnetic permeable material, which causes neural stimulation of the tissue near the magnetic permeable material. The material properties, atomic makeup and dipole alignment are all important and need to be aligned with the external device and resulting magnetic field to create the environment in which the magnetic field is boosted and sufficient to create a current density capable to stimulating the tissue adjacent to the microstimulator.

In an embodiment, the external device contains an inductive coil, that is configured to produce the one or more corresponding magnetic fields to interact with the highly magnetic permeable material within the microstimulator. The number of wire turns, the overall geometry and orientation of the inductive coil will be configured to interact with the material to create the right magnetic field to achieve stimulation at the microstimulator. The inductive coil does not need to be touching the skin to create the magnetic field within the body. The coil in this configuration is designed to be placed near the microstimulator without touching the skin. In one embodiment, one inductive coil is used to interact with more than one microstimulator. In this configuration bilateral microstimulator are placed within the PPF, and one external inductive coil is used to interact with both microstimulators. In other embodiments, one coil is used to control one microstimulator. In this embodiment, the amplitudes of stimulation can be tailored per microstimulator, which cannot happen in the case of just one externally generated magnetic field.

The amplitude of stimulation is proportional to the strength of the resulting magnetic field at the magnetic permeable material. The stimulation amplitude can be adjusted by adjusting the strength of the field generated by the external device. The electrical stimulation waveform is created by pulsing and modulating the external generated magnetic field. Standard methods can be used to create the waveforms, for example sinusoidal waveforms, but other waveforms, such as asymmetric waveforms, can be utilized as necessary.

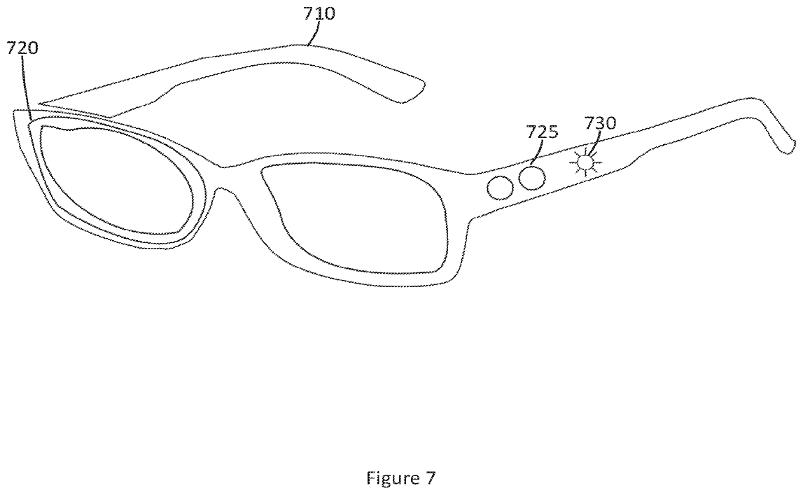

Turning to FIG. 7, In an embodiment, the external controller is configured to be worn by the patient, a wearable device. In this configuration, the external controller may be configured to be worn like a pair of glasses 710. In this embodiment, the glasses would contain all the necessary elements to generate the magnetic field and other electronics to control and manipulate the microstimulators. In this embodiment, as well as in others, the glasses will also be configured to have the inductive coils 720 placed near the microstimulators, such as the rim around the lens on either side of the glasses could contain the inductive coil 720. The nose pads may be made of any suitable biocompatible material, including but not limited to, polymers, co-polymer and/or plastics. The nose pads are configured and positioned on the glasses so that they can be manipulated to provide comfort to the subject when wearing the glasses. It should be recognized that any configuration of glasses can be used, as long as, the necessary electronics can be embedded. In this embodiment, as well as in other embodiments, bilateral stimulation can be achieved. In this embodiment, one microstimulator is inserted into the left and one in the right PPF of the subject, and the external controller (glasses) allow for simultaneous stimulation bilaterally through individual control of the magnetic fields generated on either side. In this embodiment, the glasses will contain mechanisms to allow the subject to control the amount and duration of stimulation. The glasses can have input buttons 725 that allow the subject to control stimulation amplitude, pulse width, frequency and/or stimulation duration. In one embodiment, the glasses will also provide visual, audio or haptic feedback to the user, such as through indicator light 730. The feedback can include, but not limited to, when the stimulation has started, the power level of stimulation, duration of stimulation, etc. In another embodiment, the glasses will contain onboard memory, to store data about the use of the system. The stored data may include, stimulation parameters, duration, power efficiencies, biofeedback signals, subject input, etc. These data can be wirelessly transmitted to a smart device from the glasses (controller) and subsequently uploaded to a central secure cloud-based server. Alternatively, the glasses may transmit directly to the cloud-based server.

In an embodiment, the spread of the magnetic field produced by the external controller can be reduced, which will consequently increase the amount of magnetic field directed toward the microstimulator and reduce overall power requirements for the external device. This could be achieved by configuring the stimulation coil with a ferromagnetic material shielding on one side (namely the side facing away from the body). The presence of the material will dampen the magnetic field on the side where the material is placed and hence direct more of the magnetic field to the body.

In other embodiments, the external controller can be configured to be secured to the skin of the subject. The resulting patch will contain the necessary electrodes as described above but configured to stick to the subject's skin and be worn on the cheek of the patient. In other embodiment, a double or single patch controller is configured to be wireless controlled from a smartphone, smart watch, or other Bluetooth or otherwise enabled system. In this embodiment, the smart device controls the stimulation therapy power, duration, etc. Also, in the embodiment, the one or more patches placed on the skin can include a rechargeable battery. The battery is configured to be able to apply enough power for a one or more sessions of stimulation. In many of the example therapeutic applications, the patient would not need to apply continuous, 24-hour, therapy, but instead use short duration therapy to treat acute needs, as well as period application of therapy for preventive effects. In one embodiment, the external controller is configured to be sized and shaped like a small electronic key fob or wearable watch.

In general, the closer the microstimulator is placed relative to the nerve, nerve fiber, group of nerve fibers or ganglion to be stimulated, the lower the power consumed by the wearable, which can prolong the battery life or reduce battery size in the external device. Some neuromodulation systems require certain nerve fibers to be stimulated relative to other nerve fibers to achieve the desired effects. In one embodiment, the microstimulator and external device is configured to stimulate the SPG, which contains nerve fibers passing through, of different caliber and different thickness of myelination, as well as parasympathetic neurons. In this configuration, the microstimulator can be configured to stimulate the larger parasympathetic neurons and fibers using current densities and configuration that allow for focused application to the entire SPG. This will cause the larger fibers and neurons to be activated first over the smaller and less myelinated sympathetic fibers.

In one embodiment, in which the stimulation coil is to be configured as a wearable product, flatten coil geometries would permit easier use and increased adoption. In these configurations, the stimulation coil can be made from either a rigid or flexible circuit board. The rigid material could be the industry standard FR4 or may be glass or plastic. The flexible material could be polyimide, or be BoPET, polyethylene, polyurethane, nylon or PTFE. The material selected should achieve the desired flexibility to follow the contour of the wearable device, but strong enough for multiple applications. In these configurations, the windings of the coil on one side are facing the body, and the microstimulator can be parallel to the coil. This portion of the coil facing the microstimulator produces a magnetic field that reaches into the body. The magnetic field can be made stronger if the magnetic field from the rest of the coil is contained by a ferromagnetic material.

In other embodiments, the external device (e.g., controller) is also configured to include the necessary electronics to capture informatics data from the user and from the one or both microstimulators. These data may include, but are not limited to, duration of use per stimulation session (therapy session), stimulation amplitude used during therapy, pulse width, frequency, or other electrical stimulation parameters that may be useful to collect per therapy session.

In one embodiment, the controller may also include a patient interactive and feedback system. This system may be a combination of visual, hearing or touch/pressure sensors, and/or a touchscreen. The feedback may be visual, audio, or vibration/touch/pressure feedback. The feedback system may also include alerts to the user as well during therapy. For example, the alerts can include, 50% of max amplitude has been reached, 100% of max amplitude has been reached, the duration of stimulation is 50% complete, 100% complete. In another example, the user may be alerted that the position of the controller has moved and that it needs to be realigned to provide ongoing therapy to achieve optimal coupling between the external magnetic field and the microstimulator.

In another embodiment, the controller will automatically control therapy. In this embodiment, the user will use the feedback system described above to place the controller correctly. The controller will then automatically ramp the therapy signal until it reaches a pre-determine level that is known to be comfortable and effective for the subject.

In other embodiment, the external controller can be configured as a pair of glasses, the glasses can include a smart screen that is located within the field of vision for the subject. The smart screen can provide ongoing data related to the use the system. In one embodiment, the screen provides the duration of therapy and the level of therapy for each microstimulator during use. In one embodiment, the subject is suffering from dry eye. In this embodiment, the glasses may include an IR camera, a Doppler system or other means of sensing the amount of tear film on the surface the eye. The biofeedback is that used to provide close loop therapy to the subject, in which therapy is turned on as needed. It should be appreciated that other method of biofeedback can be used as the control signal for the close loop system.

Turning to FIG. 8, In another embodiment, the microstimulator system includes a physician programmer. The external controller can be in the form of a pair of glasses 710, with the microstimulator 310 inserted into the living body. The physician programmer 810 can be configured as a tablet, a personal computer, a smartphone appliance, a tablet application or other suitable means. The physician programming allows the physician to modulate the therapy for each patient. In one embodiment, the physician programmer 810 allows the physician to individually determine the best stimulation parameters for each microstimulator independently by enabling the physician to only activate the magnetic field for each microstimulator one at a time. In another embodiment, the physician programmer 810 is configured to allow the physician to input, save, store and recall information about a specific patients' therapy into/from a secure database. In one embodiment, the physician programmer 810 is configured to wirelessly communicate with the external device. In another embodiment, the physician programmer 810 has an isolated wired link to the external device. In one embodiment, the external device allows the subject to choose from 4 to 5 different therapy levels. In this embodiment, the physician may determine and set the therapy level that produces the first onset response of therapy. The physician will then determine and set the maximum therapy the subject can tolerate. The physician programmer 810 will then automatically set the 4 or 5 therapy options ranging from low to high and program those therapy options into the controller for the subject to use independently of the programmer.

Several potential applications for this invention are disclosed, however, it is to be appreciated that the scope of the invention is not limited to the to the application solely described within the invention but can have wide ranging applications for the treatment of many disease states.

In one application of the invention relates generally to the devices and methods for stimulation of the sphenopalatine ganglia, the sphenopalatine nerve, the vidian nerve, the greater and/or deep petrosal nerves or other nerves and or branches of the sphenopalatine ganglion.

Headaches are one of the most common ailments and afflict millions of individuals worldwide. The specific etiology of headache may be difficult to pinpoint. Known sources of headache pain include trauma and vascular, autoimmune, degenerative, infectious, drug and medication-induced, inflammatory, neoplastic, metabolic-endocrine, iatrogenic (such as post-surgical), musculoskeletal and myofascial causes.

Diagnosis of headache pain will typically include an identification of one or more categories of headaches. Thera are a variety of different headaches with different features. These include, migraine headaches, including migraine headaches with aura, migraine headache without aura, menstrual migraines, migraine variants, atypical migraines, complicated migraines, hemiplegic migraines, transformed migraines, and chronic daily migraines; episodic tension headaches; chronic tension headaches; analgesic rebound headaches; episodic cluster headaches, chronic cluster headaches; cluster variants; chronic paroxysmal hemicrania; hemicrania continua; post traumatic headache; post-traumatic neck pain; post-herpetic neuralgia involving the head or face; pain from spine fracture secondary to osteoporosis; arthritis pain in the spine, headache related to cerebrovascular disease and stroke; headache due to vascular disorder, reflex sympathetic dystrophy, cervicalgia (which may be due to various causes, including, but not limited to, muscular, discogenic, or degenerative, including arthritic, posturally related, or metastatic); glossodynia, carotidynia; cricoidynia; otalgia due to middle ear lesion; gastric pain; sciatica; maxillary neuralgia; laryngeal pain, myalgia of neck muscles; trigeminal neuralgia (sometimes also termed tic douloureux); post lumbar puncture headache; low cerebro-spinal fluid pressure headache; temporomandibular joint disorder; atypical facial pain; ciliary neuralgia; paratrigeminal neuralgia (sometimes also termed Raeder's syndrome); petrosal neuralgia; Eagle's syndrome; idiopathic intracranial hypertension; orofacial pain; myofascial pain syndrome involving the head, neck, and shoulder, chronic migraneous neuralgia, cervical head ache; paratrigeminal paralysis; sphenopalatine ganglion neuralgia (sometimes also termed lower-half headache, lower facial neuralgia Syndrome, Sluder's neuralgia, and Sluder's syndrome); carotidynia; Vidian neuralgia; and causalgia; or a combination of the above.

Movement disorders treatable according to an embodiment of the disclosure may be caused by conditions including, but not limited to Parkinson's disease; cerebropalsy; dystonia; essential tremor; and hemifacial spasms. Epilepsy treatable according to an embodiment of the disclosure may be, for example, generalized or partial. Cerebrovascular disease treatable according to an embodiment of the disclosure may be caused by conditions including, but not limited to aneurysms, strokes, vasospasm and cerebral hemorrhage. Autoimmune diseases treatable according to an embodiment of the disclosure include, but are not limited to, multiple sclerosis. Autonomic disorders according to an embodiment of the disclosure may be caused by conditions including, but not limited to: gastrointestinal disorders, including but not limited to gastrointestinal motility disorders, nausea, vomiting, diarrhea, chronic hiccups, gastroesophageal reflux disease, and hypersecretion of gastric acid; autonomic insufficiency; excessive epiphoresis; excessive rhinorrhea; and cardiovascular disorders including but not limited to cardiac dysrhythmias and arrythmias, hypertension, and carotid sinus disease. Urinary bladder disorders treatable according to an embodiment of the disclosure may be caused by conditions including, but not limited to spastic or flaccid bladder. Abnormal metabolic states treatable according to an embodiment of the disclosure may be caused by conditions including, but not limited to hyperthyroidism or hypothyroidism. Disorders of the muscular system treatable according to an embodiment of the disclosure include, but are not limited to, muscular dystrophy and spasms of the upper respiratory tract and face. Neuropsychiatric disorders treatable according to an embodiment of the disclosure may be caused by conditions including, but not limited to depression, Schizophrenia, bipolar disorder, and obsessive-compulsive disorder.

In addition to the above, most individuals will have a problem with their eyes at some point in their lives. Most eye problems are not serious and do not require the care of a doctor. For example, certain ocular disorders can cause excessive tearing, chronic swelling or inflammation of eyelids, deficient tear production, or abnormal tear composition. Certain eye diseases, however, are serious and can result in blindness if left untreated.

The tears produced by the human eye are composed of three layers: the outer oily layer; the middle watery layer; and the inner mucus layer. Dry eye syndrome (also known as keratoconjunctivitis, keratitis sicca and xerophthalmia) is often used to describe a condition in which not enough tears are produced, or tears with the improper chemical composition are produced. Symptoms of dry eye syndrome vary in different people, but the following are commonly experienced by those whose tear production is inadequate: irritated, scratchy, dry or uncomfortable eyes; redness of the eyes; a burning sensation of the eyes; a feeling of a foreign body in the eye; blurred vision; excessive watering; and eyes that seem to have lost the normal clear glassy luster. Excessive dry eye can damage eye tissue and possibly scar the cornea, thereby impairing vision. These conditions can be treated according to an embodiment of the disclosure.

Blepharitis is a chronic or long-term inflammation of the eyelids and eyelashes. Among the most common causes of blepharitis are poor eyelid hygiene, excess oil produced by the glands in the eyelids, bacterial infection, and allergic reaction. There are two ways in which blepharitis may appear. The most common and least severe, seborrheic blepharitis is often associated with dandruff of the scalp or skin conditions (e.g., acne). It usually appears as greasy flakes or scales around the base of the eyelashes and as a mild redness of the eyelid. Sometimes, it may result in a roughness of the tissue that lines the inside of the eyelids, or in chalazia, which are nodules on the eyelids. Acute infection of the eyelids can result in styes. Ulcerative blepharitis is a less common, but more severe condition that may be characterized by matted, hard crusts around the eyelashes, which leave small sores that may bleed or ooze when removed. There may also be a loss of eyelashes, distortion of the front edges of the eyelids, and chronic tearing. In severe cases, the cornea may also become inflamed.

Epiphora is the term commonly used to describe a watery eye. More specifically, lacrimation describes persistent welling of tears in the eye, and epiphora is when these tears spill over onto the face. Epiphora is caused by overproduction of tears and/or inadequate/blocked drainage. Both lacrimation and epiphora can be associated with interference in vision, and the surrounding skin can become very sore and excoriated from the constant wiping of tears. An embodiment of the disclosure can be applied to treat these disorders and diseases. A microstimulator as described in this invention may be used to treat or be used as a diagnostic for several neurological diseases or disorders as noted above. In other embodiments, the microstimulator system describe may be used to overcome cosmetic problems such as, reducing skin redness, blushing, or rosacea.

The above Detailed Description of examples of the technology is not intended to be exhaustive or to limit the technology to the precise form disclosed above. While specific examples for the technology are described above for illustrative purposes, various equivalent modifications are possible within the scope of the technology, as those skilled in the relevant art will recognize.

What is claimed is:

1. A system for stimulating nerves in a living body, comprising:
an implantable microstimulator, configured to be positioned at least partially within tissue of the living body, comprising a magnetic focal element which is electrically isolated from the tissue; and
a portable external controller configured to be worn on and external to the living body, comprising a processor, a power source and an inductive coil;
wherein the processor is configured to control the power source and inductive coil to produce a time-varying magnetic field extending to the implantable microstimulator positioned within the tissue;
wherein the magnetic focal element is configured to increase the time-varying magnetic field in a space proximate to the magnetic focal element; and
wherein the increased time-varying magnetic field causes an elevated electrical current density in a stimulation area of the tissue proximate to the implantable microstimulator, causing neural stimulation within the stimulation area of the tissue.

2. The system of claim 1, where the system is configured to treat one of headaches, movement disorders and ocular disorders.

3. The system of claim 1, wherein the portable external controller comprises a plurality of inductive coils.

4. The system of claim 1, wherein the time-varying magnetic field is a periodic waveform with varying amplitude.

5. The system of claim 1, wherein the magnetic focal element has a relative magnetic permeability over 150μ.

6. The system of claim 1, wherein the magnetic focal element comprises one of iron, silicon iron, permallowy, hipernik, supermalloy, mumetal, permendur, hipereo, metglas, ferrite, nickel, and supermendur.

7. The system of claim 1, wherein the portable external controller further comprises a communication device configured to receive configuration data to be used by the processor to produce the time-varying magnetic field.

8. The system of claim 1, configured to stimulate the sphenopalatine ganglion.

9. The system of claim 1, wherein the portable external controller is configured as a pair of glasses.

10. The system of claim 1, wherein the implantable microstimulator further comprises a conductive electrode.

11. A method for stimulating nerves in a living body, comprising:
producing a time-varying magnetic field from a portable external controller configured to be worn on and external to the living body, the portable external controller comprising a processor, a power source and an inductive coil, the processor being configured to control the power source and inductive coil to produce the time-varying magnetic field, the time-varying magnetic field extending to an implantable microstimulator positioned at least partially within tissue of the living body;
wherein the implantable microstimulator comprises a magnetic focal element which is electrically isolated from the tissue;
wherein the magnetic focal element increases the time-varying magnetic field in a space proximate to the magnetic focal element; and
wherein the increased time-varying magnetic field causes an elevated electrical current density in a stimulation area of the tissue proximate to the implantable microstimulator, causing neural stimulation within the stimulation area of the tissue.

12. The method of claim 11, where the method is utilized to treat one of headaches, movement disorders and ocular disorders.

13. The method of claim 11, wherein the portable external controller comprises a plurality of inductive coils.

14. The method of claim 11, wherein the time-varying magnetic field is a periodic waveform with varying amplitude.

15. The method of claim 11, wherein the magnetic focal element has a relative magnetic permeability over 150μ.

16. The method of claim 11, wherein the magnetic focal element comprises one of iron, silicon iron, permallowy, hipernik, supermalloy, mumetal, permendur, hipereo, metglas, ferrite, nickel, and supermendur.

17. The method of claim 11, wherein the portable external controller further comprises a communication device configured to receive configuration data to be used by the processor to produce the time-varying magnetic field.

18. The method of claim 11, utilized to stimulate the sphenopalatine ganglion.

19. The method of claim 11, wherein the portable external controller is configured as a pair of glasses.

20. The method of claim 11, wherein the implantable microstimulator further comprises a conductive electrode.
